(19) United States
(12) Patent Application Publication
(10) Pub. No.: US 2017/0080373 A1
(43) Pub. Date: Mar. 23, 2017

(54) AIR PURIFICATION SYSTEM

(71) Applicant: Rolf Engelhard, Prescott, AZ (US)

(72) Inventor: Rolf Engelhard, Prescott, AZ (US)

(21) Appl. No.: 15/273,607

(22) Filed: Sep. 22, 2016

Related U.S. Application Data
(60) Provisional application No. 62/222,010, filed on Sep. 22, 2015.

(51) Int. Cl. B01D 46/42; A61L 9/20; B01D 46/44

(52) U.S. Cl. CPC B01D 46/429; B01D 46/446; B01D 46/448; B01D 46/442; A61L 9/205; B01D 2273/30; B01D 2279/50; A61L 2209/111; A61L 2209/14

(57) ABSTRACT

The systems and methods described herein are directed to an air purification system that can purify air that is passed through the air purification system. The air purification system can include one or more sensors that can detect various characteristics associated with the air that passes through the air purification system. Some implementations of the air purification system can include wireless communication capabilities that allow at least the sending of warnings to remote locations, such as a user's mobile device. In addition, the user can remotely monitor sensed data collected by the air purification system, such as via an app downloaded onto the user's mobile device. In addition, one or more settings of the air purification system can be directly or remotely adjusted (e.g., via the user's mobile device).

### CROSS-REFERENCE TO RELATED APPLICATION

The current application claims priority under 35 U.S.C. §119(e) to U.S. Provisional Patent Application Ser. No. 62/222,010, filed on Sep. 22, 2015 and entitled “AIR PURIFICATION SYSTEM,” which is incorporated by reference herein in its entirety.

### TECHNICAL FIELD

The subject matter described herein relates to an air purification system that includes one or more sensors, a warning system, and wireless communication capabilities.

### BACKGROUND

The air used to air condition structures (i.e., houses, buildings) can originate from either the inside of a structure or outside of a structure. Some problems associated with using air from outside a structure to air condition the indoor areas of a structure include the introduction of outdoor contaminants and particulates commonly found in outdoor air. Outdoor air may contain smoke and smog, which can contain carbon monoxide, ozone, and other pollutants that may irritate a person's respiratory system. In addition, introduction of mold spores and pollen, which are common particulates found in outdoor air, may cause unwanted mold to grow inside and induce allergic reactions to persons occupying the structure. In addition to the air contaminant that may be brought into a building from the outside, air contaminants may leak from a basement (i.e., through a crawl space) and accumulate in areas commonly occupied by people. Air escaping a basement may carry mold spores and potentially harmful gases, such as radon, which can pose health risks for those occupying the structure.

In addition, most structures generally “breathe” due, at least in part, to changes in outside air pressure relative to air pressures within structures. For example, when air pressure outside of a structure is greater than the air pressure within a structure, the outside air tends to leak into the structure. When air pressure outside a structure is less than the air pressure within a structure, the air inside the structure tends to leak out of the structure. Generally, the pressure differential between the outside of a structure and the inside of a structure may be caused by any number of factors (i.e., atmospheric changes, wind, exhaust fans running, stoves and fireplaces in operation, etc.). The continual “breathing” of a structure may be essential for supplying fresh oxygen to occupants of a structure. However, if air leakage into a structure is uncontrolled, the air brought into a structure may bring in undesirable contaminants and particulates that eventually may be inhaled by occupants.

Some conventional air purification systems that are currently available re-circulate the air within the structure, which prevents total indoor air purity to be achieved for at least the reasons described above. In addition, some air purification systems expel harmful byproducts, such as ozone, into the air of structures as a result of their air purification processes. Ozone is a harmful air pollutant that can be harmful to breathe, and long-term exposure to ozone may permanently reduce a person's breathing ability. In particular, children, the elderly, and people with respiratory diseases can be especially sensitive to ozone inhalation. Therefore, for at least the reasons described above, there is a need for an air purification system that can supply purified air to the inside of a structure without expelling unhealthy levels of ozone into the structure.

### SUMMARY

Various implementations of air purification systems are described herein that purify air passed through the air purification system. In one implementation, the air purification system includes a housing having an air inlet and an air outlet. The air purification system can further include a fan actuated by a control circuit that controls a rate of airflow through the air purification system and a filter for filtering out particulates from the air passing through the housing. The air purification system can further include an ultraviolet light source providing ultraviolet light to the air passing through the housing and at least one photo-catalytic element positioned adjacent the ultraviolet light source. In addition, the air purification system can include a chemical catalyst element that is exposed to the air passing through the housing and a sensor for collecting sensed data defining one or more characteristic associated with the air passing through the housing.

In some variations one or more of the following features can optionally be included in any feasible combination. The air purification system can further include a processor configured to compare the sensed data with an acceptable range. The air purification system can further include a warning system that is configured to provide an alarm to a user when the processor determines that the sensed data is not within the acceptable range. The air purification system can further include a wireless communication feature that is in communication with at least one of the processor and the warning system. The wireless communication feature can be configured to send at least one of the alarm, the sensed data, and a setting of the air purification system to a remote device. The remote device can include at least one of a mobile device and a computer. The wireless communication feature can be configured to receive an instruction from the remote location, the instruction comprising a change to the setting of the air purification system. The processor can be further configured to change a setting of the air purification system based on the comparison of the sensed data. The sensor can include a temperature gauge configured to collect sensed data defining a temperature of the air passing through the air purification system. The sensor can include a smoke detector configured to collect sensed data defining an amount of smoke in the air passing through the air purification system. The sensor can include a carbon monoxide detector configured to collect sensed data defining an amount of carbon monoxide in the air passing through air purification system.

In another interrelated aspect of the current subject matter, a method includes sensing, with a first sensor, a first characteristic of air adjacent a first side of a housing of an air purification system, the air purification system being configured to purify air passing through the housing. The method can further include determining, by a processor of the air purification system, whether the first characteristic is within an accepted first range. In addition, the method can include changing, when the first characteristic is determined to not be within the accepted first range, a setting associated with the air purification system to assist the first characteristic with falling within the accepted first range.

Some variations of the method can include sensing, with a second sensor, a second characteristic of air adjacent a second side of the housing of the air purification system and calculating, by the processor, a difference between the first characteristic and the second characteristic. In addition, the method can include determining, by the processor, if the calculated difference is within an accepted second range and changing, when the calculated difference is determined to not be within the accepted second range, the setting associated with the air purification system to assist the calculated difference with falling within the accepted first range. The method can include setting includes a fan speed of a fan configured to control a speed at which the air passes through the housing. The first characteristic can include a temperature, a pressure, an amount of smoke in the air, and an amount of carbon monoxide in the air. The method can further include activating, based on the determining, a warning system of the air purification system. The activating the warning system can include at least one of activating an audible alarm and sending an alert to a remote device. The method can include sending, from a wireless communication feature of the air purification system in wireless communication with a remote device, information related to at least one of the first characteristic and the second characteristic to the remote device. In addition, the method can include receiving, at the wireless communication feature, a setting instruction from the remote device and changing, based on the setting instruction, the setting of the air purification system. The second side of the housing can be located outside of a structure to which the air purification system is coupled to and the first side of the housing is located at least one of inside the housing and inside the structure to which the air purification system is coupled to.

Systems and methods consistent with this approach are described as well as articles that comprise a tangibly embodied machine-readable medium operable to cause one or more machines (e.g., computers, etc.) to result in operations described herein. Similarly, computer systems are also described that may include a processor and a memory coupled to the processor. The memory may include one or more programs that cause the processor to perform one or more of the operations described herein.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

These and other aspects will now be described in detail with reference to the following drawings.

FIG. 1 illustrates an embodiment of an air purification system that includes sensors, a processor, a warning system, and a wireless communication feature.

FIG. 2 illustrates a flow chart of a pressure differential function of the air purification system.

FIG. 3 illustrates a flow chart of a heating function of the air purification system.

FIG. 4 illustrates a flow chart of a cooling function of the air purification system.

FIG. 5 illustrates a flow chart of a method of sensing carbon monoxide levels and activating the warning system when carbon monoxide levels are sensed to be at an unsafe level.

FIG. 6 is a cut-away view of a high intensity air purifier in accordance with preferred implementations.

FIG. 7 is an exploded view of a high intensity air purifier in accordance with preferred implementations.

FIG. 8 shows a star pattern chamber.

FIGS. 9A and 9B illustrate a continuous helical ramp chamber.

FIG. 10 illustrates a modular ramp chamber.

FIG. 11 illustrates radial louvers that inhibit UV light from exiting the chamber.

FIG. 12 illustrates the high intensity air purifier of FIG. 6 in including a processor, sensors, a warning system and a wireless communication feature.

FIG. 13 is a cut-away view of a high intensity air purifier in accordance with an alternative implementation.

Like reference symbols in the various drawings indicate like elements.

### DETAILED DESCRIPTION

This document describes implementations of air purification systems that purify air that is passed through the air purification system. In some implementations, the air purification system can include one or more sensors that can detect various characteristics (e.g., air quality, temperature, carbon monoxide levels, etc.) associated with the air that passes through the air purification system. The air purification system can also include a processor that analyzes the sensed data collected by the one or more sensors, such as compare the collected data against defined ranges of acceptable values.

Some implementations of the air purification system can also include a warning system that can alert a user when the processor determines that the sensed data is not within a defined range of acceptable values (e.g., the level of carbon monoxide is too high). The alerts can be made locally at the air purification system or remotely, such as at a user's mobile device. As such, some implementations of the air purification system can include wireless communication capabilities that allow at least the sending of warnings to remote locations, such as the user's mobile device. In addition, the user can remotely monitor the sensed data collected by the air purification system. In some implementations, the air purification system can be automatically and dynamically adjusted (e.g., fan speed) based on the collected sensed data. In addition, the air purification system can be directly or remotely (e.g., via the user's mobile device) adjusted.

The air purification systems can be configured to purify air passing through the air purification system to at least a level that is generally healthy for human inhalation. In addition, the air purification system may function to provide warmer or cooler purified air to the inside of a structure. Furthermore, the air purification system may include a feature to purify circulated air to the inside of a structure. In alternative implementations, an air purification system may also include a solar heating element that can function to increase the temperature of the air purified in the air purification system.

In some implementations, the air purification system includes features that allow it to be integrated into a structure and provide an airflow pathway between the outside (i.e. “outside space”) and inside (i.e. “inside space”) of the structure. The air purification system may be coupled to an air duct or pipe that is already part of the structure so that installation of the air purification system generally does not require additional holes or penetrations into any walls of the structure. Alternatively, generally any wall of a structure may be penetrated in order to adapt an air purification system to the structure. In general, the air purification system may be integrated into a structure so that it can purify air as it is forced from the outside of the structure into the inside area of the structure, as will be discussed in more detail below.

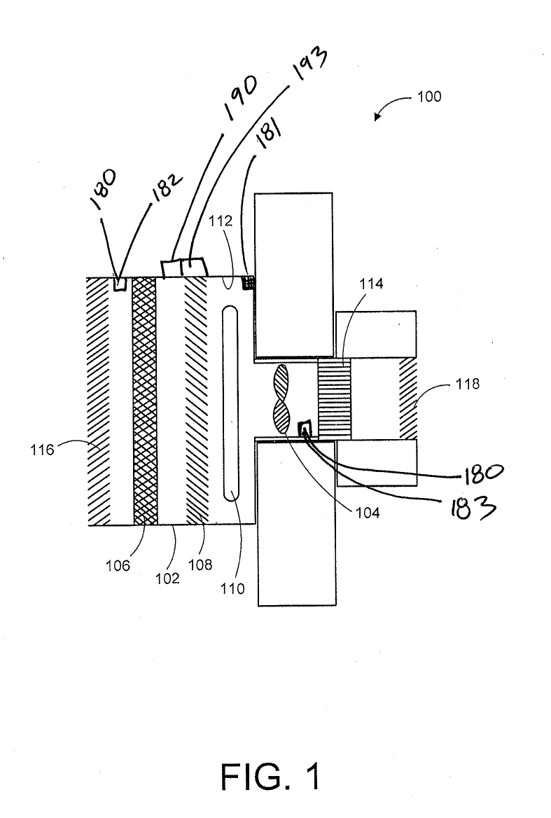

Turning now to the figures, FIG. 1 shows an implementation of the air purification system 100. The air purification system 100 can include a housing 102 that generally houses the components of the air purification system 100. The housing 102 may be formed of one or more parts and may include features (i.e., mounting holes, fasteners, etc.) that can assist in securing the placement of the air purification system 100 to a structure. In addition, the housing 102 of the air purification system 100 can accommodate a fan 104 that, when circulating, forces air to be passed through the air purification system 100. For instance, a fan 104 in the housing 102 is arranged to draw in air from the outside of a structure, force it through the air purification system 100, and expel the newly purified air into the structure. The fan 104 can be a variable speed fan such that the rate at which air is passed through the air purification system 100 can be varied. The speed of rotation of the fan 104 may be manually or remotely controlled by a user, programmed, and/or dynamically adjusted based on collected sensed data, as will be discussed in further detail below. Although described herein as a fan, any number of mechanisms may be used to force air through the air purification system 100 without departing from the scope of the present disclosure.

In general, if all structural fixtures allowing air into the building (i.e., windows, doors, etc.) are generally closed and the air purification system 100 is providing adequate airflow into the structure from the outside, the air purification system 100 may essentially become the sole source of outside air into the structure. Therefore, not only can the air purification system 100 generally provide the sole source of outside air into a structure, but it can also create and maintain a pressure differential between the inside and outside of the structure. For instance, as the air purification system 100 forces air from the outside of a structure and expels it into the inside of a structure, the air purification system 100 ultimately can cause the inside of the structure to have a higher pressure than the outside of the structure. The ability of the air purification system 100 to create and maintain this pressure differential generally limits any air entering the structure from the outside to only through the air purification system 100. Therefore, the remaining air leaks throughout the structure, which may have otherwise been a source of contaminants entering the building, are generally limited to air exiting the building. By limiting the source of airflow into the structure to generally solely being through the air purification system 100, the reduction in outside contaminants (i.e., mold spores, pollen, dust, smoke, smog etc.) entering the inside of the structure can be reduced due to the air purification system's 100 ability to eradicate air contaminants as the air is passed through the air purification system 100, as will be described in more detail below. Ultimately, this may help reduce allergic reactions, breathing irritations and other health problems associated with exposure to air contaminants for those people occupying the structure.

The air purification system 100 may be sized, dimensioned and powered such that it can appropriately maintain clean air within an area of a structure. For example, the air purification system 100 may handle 0.5 air changes per hour, which is generally known to be the air exchange rate (AER) necessary to continuously ventilate a house under moist conditions. However, the air purification system 100 may be sized and powered to effectively maintain cleaner air in a number of sized and dimensioned structures without departing from the scope of the present disclosure.

The air purification system 100 includes air purification technology that reduces, if not eliminates, the release of ozone into the inside area of the structure to which it is providing purified air. Ozone can cause health problems, including respiratory tract irritation and breathing difficulties. Therefore, the air purification system is configured to significantly reduce, if not prevent, the release of ozone into the inside of the structure due to any air purification processes, as will be discussed below.

As illustrated in FIG. 1, the air purification system 100 includes one or more of a filter 106, photo-catalytic element 108, ultraviolet (UV) light source 110, reflective material 112, and chemical catalytic element 114. In addition, and also shown in FIG. 1, the air purification system 100 may further include a louvered screen 116 and a directional outlet 118. The air purification system 100 may be installed into a structure such that the louvered screen 116 is in generally in contact with the outside air of the structure and the directional outlet 118 is generally in contact with the inside air of the structure. In this configuration, the fan 104 can function to draw air in from the outside and force it to pass through the louvered screen 116, filter 106, photo-catalytic element 108 and become exposed to UV light. After the air is exposed to the UV light source 110, the fan 104 can continue to force the air out through the chemical catalytic element 114 and directional outlet 118 before being expelled into the inside of a structure.

In general, the louvered screen 116 provides a directional airflow inlet into the air purification system 100. Additionally, the louvered feature of the louvered screen 116 assists in reducing turbulent flow and minimizing, if not preventing, direct UV light emissions from the air purification system 100. Once air has passed through the louvered screen 116, the air is then forced through one or more filters 106, as shown in FIG. 1. Generally, the one or more filters 106 function to capture and eliminate various sized particulates from the air. In general, filters may function to capture generally larger-sized particulates. However, any number of filters may be used that are designed to capture any number of types and sizes of particulates without departing from the scope of the present disclosure.

Once the air has passed through the one or more filters 106, the air is then forced through the photo-catalytic element 108 and exposed to the UV light source 110. For example, the photo-catalytic element 108 may be comprised of a thin-film photo-catalyst, such as Titanium dioxide, that is generally coated over an element that allows air to pass through (i.e., a louvered screen). Similar to the louvered screen 116 described above, louvers may be used again here to minimize direct UV light emissions from the air purification system 100 and reduce turbulent airflow. The photo-catalyst coating enables particulates, such as organic compounds, in the air to come into contact with the photo-catalyst in order for them to be destroyed upon exposure to UV light. After the particulates have come into contact with the photo-catalyst, the particulates are exposed to the UV light source 110. As described above, the UV light source 110 activates the photo-catalyst to destroy the remaining particulates in the air. Reflective material 112 may surround at least a portion of the UV light source 110, and may function to increase the intensity of the UV light and exposure of the UV light to the particulates. Increased intensity and exposure of UV light to the particulates can increase the effectiveness in activating the photo-catalyst and eradicating the particulates from the air. In general, the combination of a photo catalyst and UV light can effectively eradicate any remaining particulates in the air the filter was unable to remove. Any number of photo-catalysts may be used to eliminate particulates from the air without departing from the scope of the present disclosure.

After the air has been exposed to the UV light source, the air is forced past the fan 104 and through a chemical catalytic element 114 before being expelled through the directional outlet 118 and into the inside of the structure. The chemical catalytic element 114 may be a screen or filter that is generally coated with a chemical catalyst. The chemical catalyst generally functions to decompose ozone that was formed as a byproduct during the air purification process conducted in the air purification system 100. As mentioned above, ozone may be hazardous to a person's health, so it is a benefit of the air purification system 100 to generally prevent the expulsion of ozone. By way of example, chemical catalysts such as those including manganese dioxide may be used to decompose ozone in the air purification system 100. However, any number of chemical catalysts may be used to cause the decomposition of ozone without departing from the scope of the present disclosure.

In addition, the directional output 118 may include slats that enable a user to direct the outflow of air from the air purification system 100 into the inside of the structure. Additionally, and shown in FIG. 1, the airflow passage way leading up to the directional output 118 may be designed and structured such that it is a generally cylindrical passageway. A generally cylindrical airflow passageway can promote laminar flow, which can ultimately provide a desirable streamline flow from the air purification system 100 into the inside of a structure. However, any number of shaped airflow passageways may be provided in the air purification system 100 that promote a laminar flow of air through the air purification system 100 without departing from the scope of the disclosure.

The air purification system 100 may further include a control circuit that may be contained within at least a part of the housing 102. For example, the control circuit may be located on the portion of the housing that is exposed to the inside of the structure. Furthermore, the control circuit can assist in providing the air purification system 100 with user-programmable features and functions conveniently accessible to a user from the inside of the structure. The control circuit may control any number of electrically powered components and features within the air purification system 100. For example, the control circuit can control the fan 104 speed in order to produce a desired rate of airflow through the air purification system 100. Additionally, the control circuit can enable the fan speed to be manually or remotely controlled by a user, or programmed to run at a certain speed or range of speeds. In addition, the control circuit can include one or more sensors that collect sensed data (i.e., pressure, temperature, etc.) and, based on the sensed data, the speed of the fan 104 can be automatically adjusted, as will be discussed in further detail below.

By way of example, the control circuit may include a pressure sensor that can collect sensed pressure data from either inside or outside of the structure. From these collect sensed pressure data, the control circuit can then either increase or decrease the fan speed, as necessary, in order to achieve a pressure differential value or range between the inside and outside of the structure. The pressure differential value or range may be set by a user, or it may be a pre-programmed setting embedded within the air purification system 100. The ability of the air purification system 100 to monitor this pressure differential enables the air purification system 100 to efficiently respond to changes in pressure within the structure, such as when a door is opened, without relying on a user.

As shown in FIG. 1, some implementations of the air purification system 100 can include one or more sensors 180 that are located in a variety of locations about the air purification system 100. The sensors 180 can sense a variety of characteristics (e.g., air quality, temperature, carbon monoxide levels, etc.) associated with either the air purification system 100 or the air that passes through the air purification system 100. In addition, the sensors 180 can send sensed data to a processor 181 associated with the air purification system 100. The processor 181 can process and analyze the sensed data and, in turn, modify one or more parameters of the air purification system 100 (e.g., fan speed, direction of air flow, etc.) in order to achieve a desired result. Additionally, the air purification system 100 can include a warning system 190 that can deliver a warning or alert to a user based on the sensed data processed by the processor 181. The sensors 180 can communicate either wirelessly or directly with the processor, and the processor can communicate either wirelessly or directly with the warning system. The warning system 190 can communicate in a variety of ways to the user, including directly from the air purification system (e.g., an audible alarm) or remotely (e.g., mobile alerts, etc.), as will be discussed in greater detail below.

For example, one of the sensors 180 can include a carbon monoxide sensor 182 that can detect the amount of carbon monoxide that is in the air that either surrounds or passes through the air purification system 100. The carbon monoxide sensor 182 can be in communication with the processor 181, and the processor 181 can receive sensed data from the carbon monoxide sensor 181, such as on a continual basis. The processor 181 can evaluate the sensed data from the carbon monoxide sensor 182 and determine when the sensed air contains an unsafe level of carbon monoxide. This can be determined by the processor 181 comparing the sensed data from the carbon monoxide sensors 182 against stored acceptable carbon monoxide level ranges. Such ranges can be, for example, set by the user. When the processor determines the amount of carbon monoxide is not within an acceptable range, the processor 181 can instruct the warning system 190 to deliver a warning to the user.

The warning system 190 can include an audible alarm located at or near the air purification system 100, which can provide an audible alarm to a user. Some implementations of the warning system can include a wireless communication feature 193 that can allow the air purification system 100 to communicate wirelessly (e.g., via text message, email, phone call, etc.) to one or more remote devices, such as a user's mobile device (e.g., phone, tablet, etc.). As such, the user can receive alerts from the warning system 190 remotely from the air purification system 100. For example, the user can receive warnings about air conditions within the user's home while away from the home.

In addition, the air purification system 100 can include wireless communication features 193 (e.g., internet access, Bluetooth, etc.) that allow a user to monitor the sensed data being collected from the sensors 180, as well as monitor and adjust settings associated with the air purification system 100. For example, the user can download an app onto the user's mobile device that allows the user to observe and monitor the air temperature (e.g., via temperature sensors 183) or amount of smoke or smog in the air that is either passing through or surrounding the air purification system 100. From the app on the user's mobile device, the user can also adjust one or more settings associated with the air purification system 100, such as the speed of the fan 104. For example, the user may want to adjust one or more settings associated with the air purification system 100 as a result of observing data collected from one of the sensors 180.

In some implementations, the air purification system 100 can dynamically and automatically adjust one or more settings associated with the air purification system 100. For example, the air purification system 100 can dynamically adjust the speed of the fan 104 based on data collected from one or more sensors 180 in order to maintain or achieve a desired air quality or characteristic. This can relieve the user from having to continually monitor the collected data readings and adjust the air purification system 100 settings, as well as allow the air purification system 100 to effectively and efficiently maintain safe and desirable air qualities, such as within office spaces and homes.

As discussed above, the air purification system 100 can include one or more sensors 180, which can include a carbon monoxide sensor, temperature sensor 183, smog detector, smoke detector, pressure sensor, etc. In addition, any number of settings associated with the air purification system 100 can be dynamically and automatically adjusted by the air purification system 100, such as in response to collected data, as well as directly or remotely adjusted by a user, such as via an app loaded onto the user's mobile device.

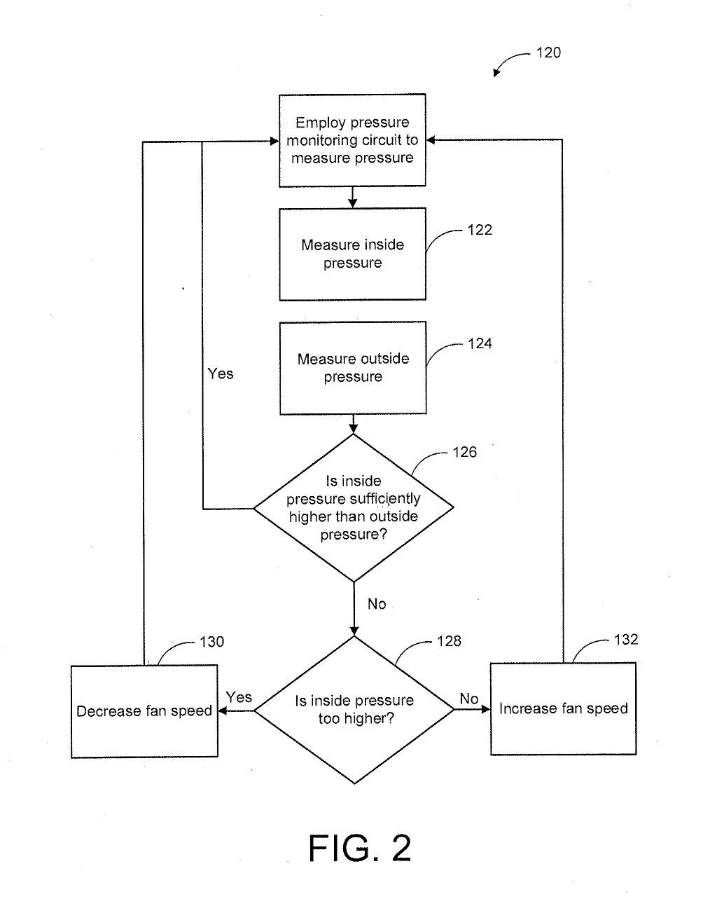

FIG. 2 is a flow chart of a method 120 for controlling an air purifier in accordance with some implementations. The method 120 can be used to determine the pressure differential existing between the outside and inside of a structure and vary the fan speed accordingly. As shown in FIG. 2, inside pressure is measured at 122, and outside pressure is measured at 124. The inside and outside pressures can be measured by one or more pressure measuring elements, such as a digital barometer or manometer. However, any number of pressure measuring elements may be employed by a pressure monitoring circuit of the air purification system 100 in order to measure at least the inside and outside air pressure of a structure. For example, a pressure measuring element employed to measure the inside air pressure of a structure may also be the same pressure measuring element that measures the outside air pressure of the structure. At 126, the method 120 further includes determining whether the measured inside air pressure is sufficiently greater than the measured outside air pressure. If the measured inside pressure is sufficiently greater than the measured outside pressure, the fan speed is generally not changed. However, if the inside air pressure is not sufficiently greater than the outside air pressure, the fan speed is changed. At 128, it is determined whether the inside air pressure is too high. At 130, the fan speed is decreased if the inside air pressure is determined to be too high. At 132, the fan speed is increased if the inside air pressure is determined to be too low. As described above, an increase in fan speed increases the air expelled into the structure by the air purification system 100, which can eventually cause the pressure within the structure to increase relative to the outside of the structure.

In addition to purifying air, the air purification system 100 may provide warmer or cooler air to the structure relative to the air temperature inside the structure. For example, the control circuit can include temperature measuring elements or sensors 180 (e.g., thermistors, thermocouples, etc.) that can measure the outside and inside air temperatures of a structure. From these measurements, the control circuit can then either increase or decrease the fan speed, as necessary, in order to achieve a defined temperature value, or range, inside the structure. The defined temperature value, or range, may be manually set by a user, or it may be a pre-programmed setting of the air purification system 100. The ability of the air purification system 100 to monitor the inside temperature of the structure enables the air purification system 100 to efficiently respond to changes in temperature within the structure, such as when a door is opened, without relying on a user. A user can also monitor the temperatures remotely, such as through an app on a mobile device that receives sensor readings 180, such as temperature readings. From the mobile device (via the app) the user can adjust one or more settings of the air purification system 100, such as the speed and airflow direction of the fan 104, in order to achieve desired temperatures surrounding the air purification system 100.

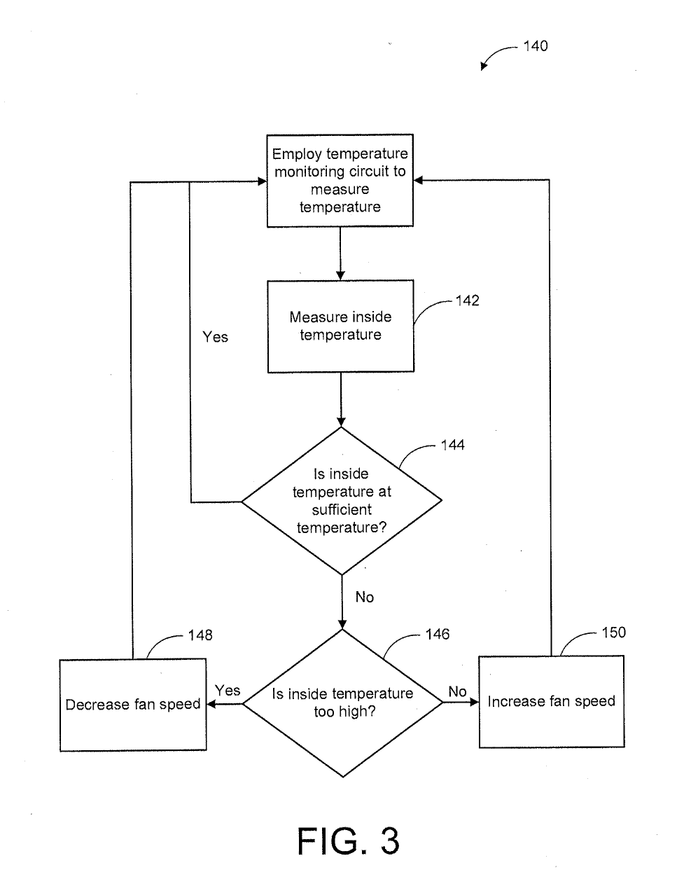

FIG. 3 is a flowchart of a method 140 for controlling temperature within a structure using an air purification system, in accordance with implementations described herein. The method 140 can be used to determine the temperature inside a structure and vary the fan speed accordingly (i.e., by the air purification system 100 or by the user either directly or remotely) in order to generally maintain warm inside air temperatures. As shown in FIG. 3, inside temperature is measured at 142. At 144, it is determined whether the inside temperature is at a desired temperature, or within a desired temperature range, which may be user defined or pre-programmed. If the measured inside temperature is at the desired temperature, or within the desired temperature range, the fan speed is generally not changed. However, if the inside air temperature is not at the desired temperature, or within the desired temperature range, the fan speed is changed. At 146, it is determined whether the inside air temperature is too high. At 148, the fan speed can be decreased if the inside air temperature is determined to be too high. At 150, the fan speed can be increased if the inside air temperature is determined to be too low. In general, this heating function only works under the conditions where the outside temperature of the structure is greater than the inside temperature of the structure.

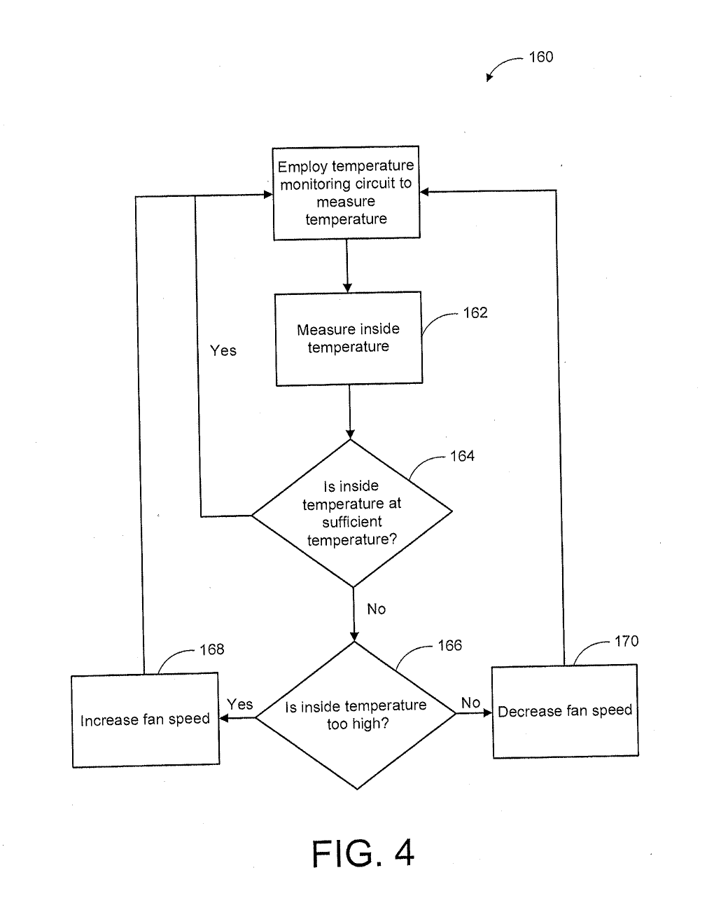

FIG. 4 is a flowchart of a method 160 for controlling temperature within a structure using an air purification system, in accordance with implementations described herein. The method 160 can be used to determine the temperature inside a structure and vary the fan speed accordingly (i.e., by the air purification system 100 or by the user either directly or remotely) in order to generally maintain cool inside air temperatures. As shown in FIG. 4, inside temperature is measured at 162. At 164, it is determined whether the inside temperature is at a desired temperature, or within a desired temperature range, which may be user defined or pre-programmed. If the measured inside temperature is at the desired temperature, or within the desired temperature range, the fan speed is generally not changed. However, if the inside air temperature is not at the desired temperature, or within the desired temperature range, the fan speed is changed. At 166, it is determined whether the inside air temperature is too high. At 168, the fan speed can be increased if the inside air temperature is determined to be too high. At 170, the fan speed can be decreased if the inside air temperature is determined to be too low. Similar to the heating function described above, the cooling function generally only works under the conditions where the outside temperature is less than the inside temperature of the structure.

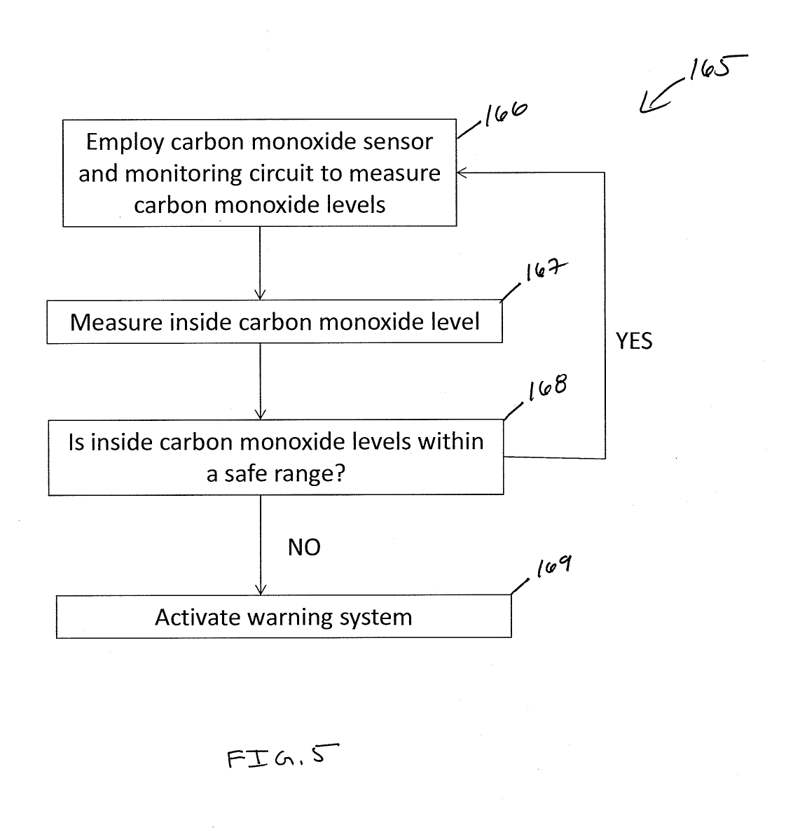

FIG. 5 is a flowchart of a method 165 for sensing carbon monoxide levels and activating the warning system 190 when carbon monoxide levels are sensed to be at an unsafe level. The method 165 can be used alert a user that is near the air purification system 100 (e.g., via an alarm associated with the air purification system 100) or remotely alert a user (e.g., via a mobile device). As shown in FIG. 4, at 166, a carbon monoxide sensor is employed, such as by a monitoring circuit associated with the processor, to measure carbon monoxide levels in the air either flowing through or surrounding the air purification system. The level of carbon monoxide is measured at 167. At 168, it is determined whether the level of carbon monoxide is within a safe range, which may be user defined or pre-programmed. If the measured level of carbon monoxide is not within the safe range, at 169, the warning system 190 can be activated. As discussed above, the warning system 190 can include an alarm associated with the air purification device 100 that, for example, can provide an audible alarm. The warning system can also include wireless communication capabilities that allow it to provide alerts to the user's mobile device(s). If the measured level of carbon monoxide is within the safe range, the warning system may not be activated, as shown in the flowchart in FIG. 5.

At least one benefit of having various sensors associated with the air purification system 100 and allowing either the air purification system 100 or a user monitor the sensed data is that since the air purification system 100 is circulating or creating a flow of air during the purification process, unsafe conditions (such as harmful levels of carbon monoxide) can be detected more quickly. As such, unsafe conditions can be made aware to a user more quickly (via the warning system 190), as well as allow either the air purification system 100 or user to remedy the unsafe condition, such as adjust a setting of the air purification system 100 (e.g., speed or direction of airflow of the fan 104).

The air purification system as described herein may be configured with a solar heating element such that the solar heating element may function to increase the air temperature at least before it is forced through the air purification system. In this configuration, the air purification system may provide heated air that has a greater temperature than both the inside and outside air temperatures of a structure. By way of example, the air purification system 100 may be installed on a south-facing part of a structure that receives solar radiation during the wintertime. In this configuration, the solar radiation would strike this south facing wall in the northern hemisphere generally only during the wintertime when heating the building is desired. Furthermore, the heating effect of the solar irradiated wall can be enhanced by painting the wall dark and covering the wall with a clear glass or plastic in order to trap at least some solar energy between the covering and the wall. In addition, the air purification system 100 may include a solar cover that may be placed adjacent the air intake, or louvered screen 116, to further enable the air purification system 100 to expel solar heated air into the structure.

In addition, some implementations of the air purification system 100 may include a re-circulation feature that can purify re-circulated air inside the structure. This re-circulation feature may include an airflow loop through the air purification system 100 that enables air from inside the structure to be drawn into the air purification system 100, and then expelled back into the inside of the structure as purified air. In addition, the re-circulation loop may be partially or fully closed at any time for enabling partial or full air re-circulation of air within the structure. In particular, the re-circulation feature may be desirable when a large temperature differential exists between the inside and outside of the structure, or when the outside air is extremely polluted. In general, a user may manually activate the re-circulation feature, or the re-circulation feature may be automatically activated by the control circuit in response to, for example, changes in outside air temperature or quality.

In another implementation of the current subject matter, the air purification system can include a high intensity air purifier (HAIP), a super oxidation purifier, and a controller for controlling operation of any of various purification systems described herein. In addition, the HAIP can include any of the functions or features described above, such as with regards to the sensors, processor, warning system, and wireless communication capabilities. As such, the HAIP can sense a variety of characteristics (e.g., air quality, temperature, carbon monoxide levels, etc.) associated with the air that passes through the HAIP. The HAIP can also include a processor that analyzes the sensed data collected by one or more sensors, such as compare the collected data against defined ranges of acceptable values.

Some implementations of the HAIP can also include a warning system that can alert a user when the processor determines that the sensed data is not within a defined range of acceptable values (e.g., the level of carbon monoxide is too high). The alerts can be made locally at the HAIP or remotely, such as at a user's mobile device. As such, some implementations of the HAIP can include wireless communication capabilities that allow at least the sending of warnings to remote locations, such as the user's mobile device. In addition, the user can remotely monitor the sensed data collected by the HAIP. In some implementations, the HAIP can be automatically and dynamically adjusted (e.g., fan speed) based on the collected sensed data. In addition, the HAIP can be directly or remotely (e.g., via the user's mobile device) adjusted.

In general, a HAIP includes an axial fan, an inlet radial louver, a reaction chamber having a UV light source, an outlet radial louver, and a photo catalyst. The axial fan moves air into and through the reaction chamber, not in a linear, but in a spiral fashion. This is due to the rotation of the fan's impeller blades. The spiral airflow around the UV light source is desirable because it creates more even exposure of all air to UV light, and it promotes spinning of the airborne particles, which gives UV exposure to all sides of the particles.

Immediately after leaving the axial fan, the moving air has to pass through the inlet radial louver. The louver blades are angled such that they further promote the spiral airflow created by the axial fan. The surface of the radial louver that is facing inward, toward the UV reaction chamber, is coated with the photo catalyst. This surface is heavily irradiated with ultraviolet light. First, the UV light comes directly from a UV lamp that is positioned perpendicular to the radial louver. Second, the UV light comes from the walls of the UV reaction chamber, which are lined with a reflective lining. The reflective lining is a “lambertian” reflector that reflects light in all directions, thereby striking the photo catalyst from all angles with massive amounts of UV.

As with the radial louver on the inlet of the UV reaction chamber, the second radial louver is located on the outlet side of the UV chamber. The second radial louver functions in the same way, and can also be coated with photo catalyst material. The second radial louver further promotes spiral flow of the air. The placement of the radial louver photo catalysts, in combination with the lambertian reflective lining of the UV reaction chamber, creates a “light tight” chamber from which no UV energy can escape unused. Radially, no UV light escapes because it is continually being reflected inward to increase the UV intensity within the chamber. Longitudinally traveling light, which would otherwise escape from the ends of the UV reaction chamber, strikes the photo catalytic surfaces on both ends where the resulting chemical reaction destroys microbial and chemical contaminants. This “light tight” construction also serves to prevent human eyes and skin from becoming exposed to harmful UV light.

One further advantage of this construction is that the radial louver in combination with the axial fan creates a turbulent airflow over the photo catalytic surfaces. Since the photo catalytic reaction only occurs directly on the photo catalyst surface, it is beneficial to create a turbulent airflow that brings all the air to this surface for a short contact period.

The outlet side of the UV reaction chamber can also house a chemical catalyst. This catalyst interacts with ozone and carbon monoxide to convert them to oxygen and carbon dioxide (among other reactions). The chemical catalytic reaction only takes place where the air touches the catalytic surfaces. Again, it is desirable to have a turbulent flow in the chemical catalyst. This is also achieved by the radial louvers, yet another advantage of this arrangement. The HAIP can be housed within a housing, which in turn can be attached to a rotating AC plug for convenient attachment to a standard wall electrical outlet. The housing can be shaped as a tube or cylinder, and have a small form factor for easy and unobtrusive deployment within a house or workspace.

High Intensity Air Purifier (HAIP)

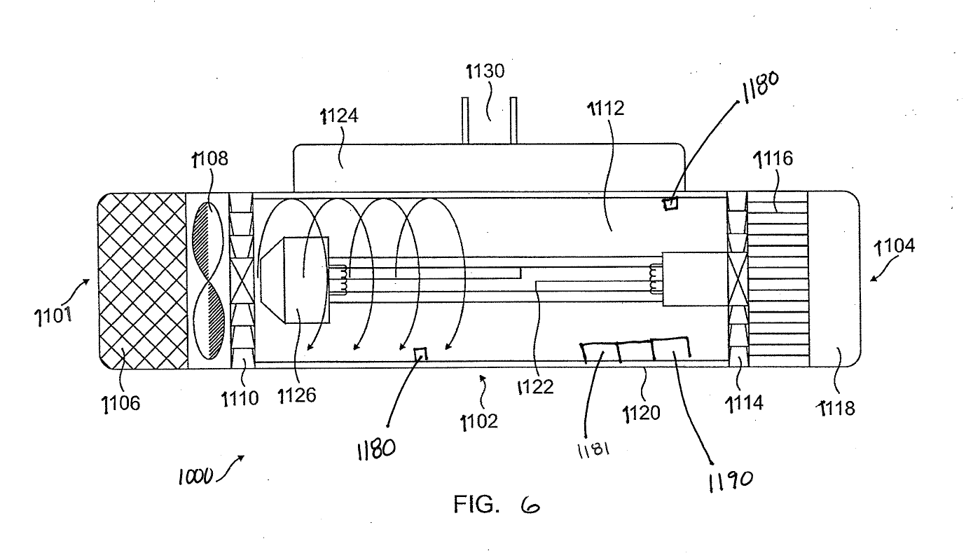

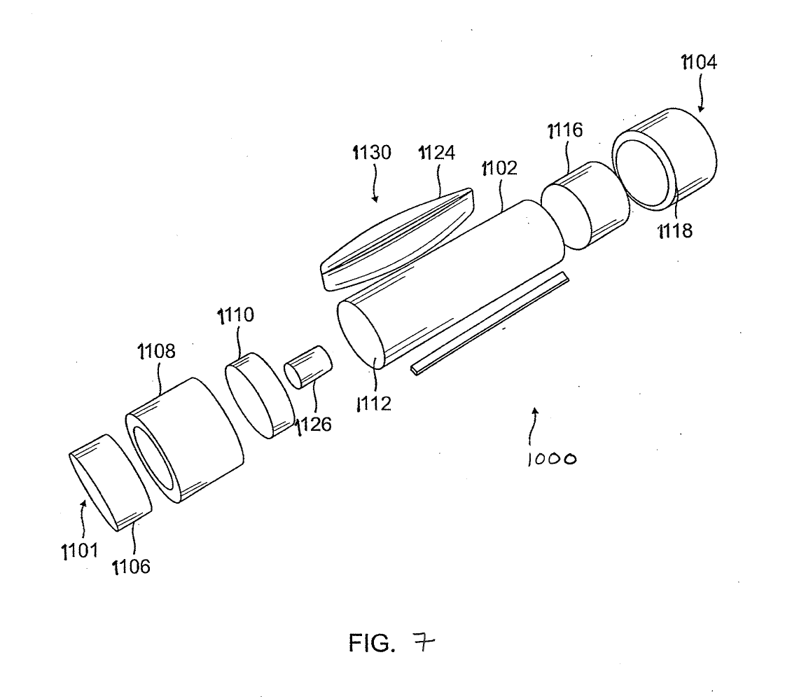

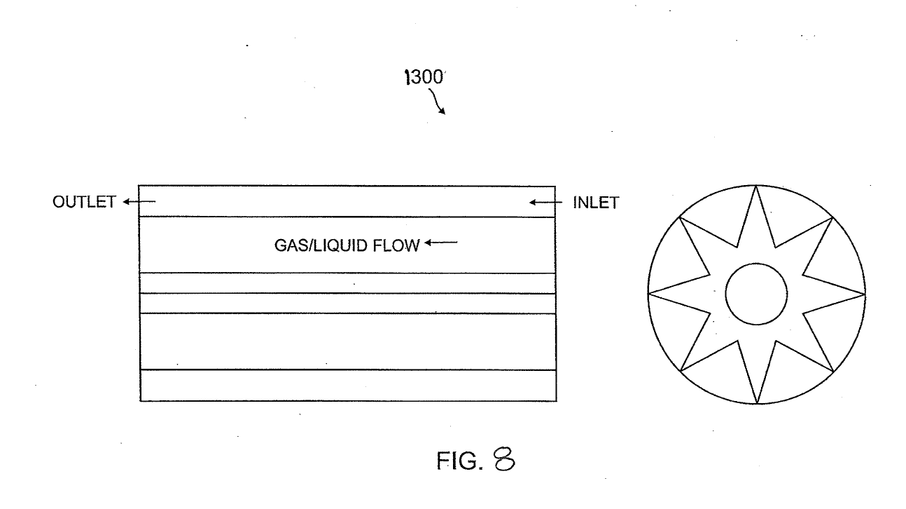

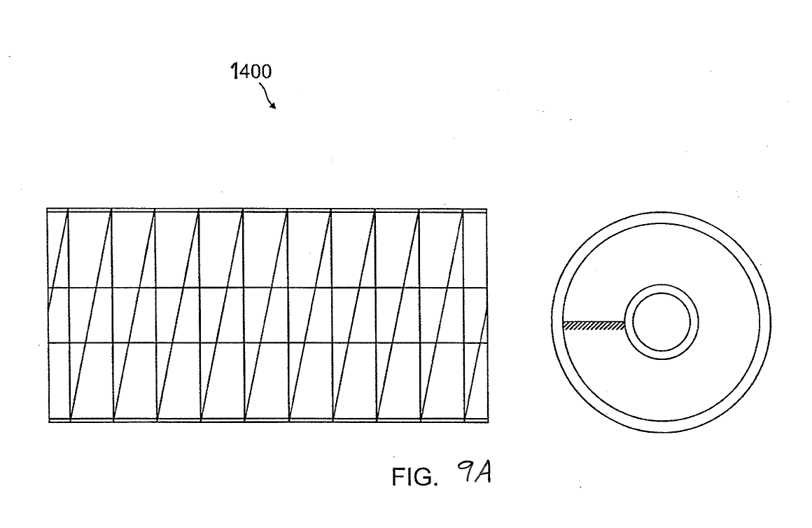

FIGS. 6 and 7 show a cross sectional view and an exploded view, respectively, of a HAIP 1000 that is preferably formed and configured to be plugged directly into a standard two- or three-pronged electrical outlet for immediate and continuous operation. The HAIP 1000 can rotate relative to the electrical outlet to change a direction in which it takes in air and discharges purified air. For instance, an inlet 1101 of the HAIP 1000 can be directed toward a source of air contamination such as a pet food dish, pet bed or litter box, or waste basket. In this way, a relative low pressure area is created around the inlet 1101, which draws in contaminated air away from the source of air contamination, where it is treated within the HAIP 1000 to reduce or eliminate particulates, odors, bacteria, viruses, etc., and the HAIP 1000 in turn discharges purified air through an outlet 1104 toward an area where clean, treated air is desirable.

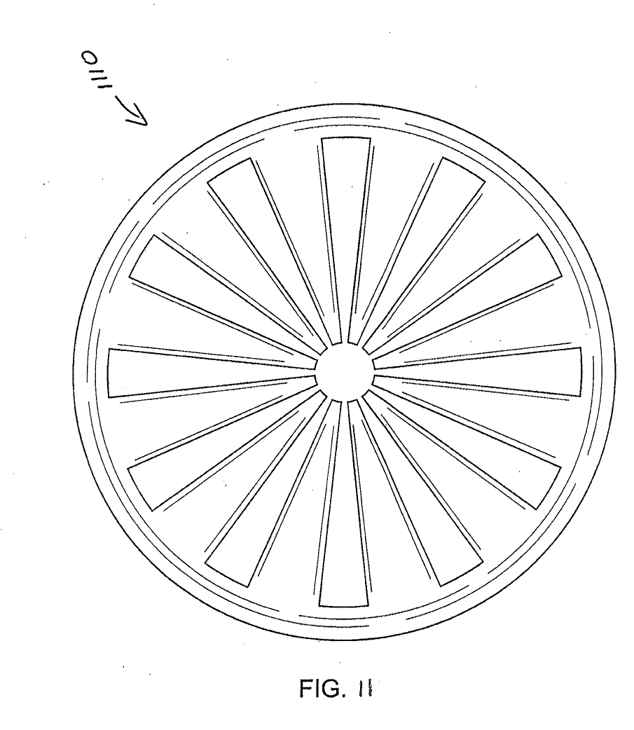

In accordance with some implementations, the HAIP 1000 includes a pre-filter 1106 connected with the inlet 1101, and an axial fan 1108 for drawing in air into the inlet 1101 and pre-filter 1106, and toward a first radial louver 1110, an example of which is shown in FIG. 11. The first radial louver 1110 is connected to an input to a reaction chamber (RC) 1112, which is part of an ultraviolet-based super oxidation purifier (SOP) system explained in more detail below. The axial fan 1108 and first radial louver 1110 provide a spiral airflow within the HAIP 1000, while also preventing a direct line of sight into the RC 1112 to prevent human exposure to harmful UV rays.

The pre-filter 1106 reduces relatively larger particulates and other air contaminants from the air drawn into the inlet 1101 before the air reaches the RC 1112. The pre-filter 1106 is preferably selectable and configurable for a particular particulate or contaminant. For example, the pre-filter 1106 can include a smoke filter, for areas where smoke is present from sources such as tobacco products, wood stoves, outside environment (brush fires, etc.) or other smoke sources. The pre-filter 1106 can include a pet filter, for areas where pet hair, feathers, dander, etc., are present. In yet other implementations, the pre-filter 1106 can include a dust and pollen filter, for areas having high pollen and/or dust contamination. The pre-filter 1106 can be configured as one or more replaceable cartridges, for addressing a particular life of each cartridge before it needs to be replaced. The pre-filter 1106 can be formed of a cleanable cartridge, such as made of a sponge-like material. In yet other implementations, the pre-filter 1106 is configured as a static filter which attracts particulates by electrostatic energy. These types of static filters can be routinely cleaned by flushing or vacuuming.

The HAIP 1000 further includes a second radial louver 1114 connected to an output of the RC 1112, a catalyst cartridge 1116 connected to the second radial louver 1114, and a post filter 1118 connected to the catalyst cartridge 1116 and which at least partly forms the outlet 1104 of the HAIP 1000. The post filter 1118 can include an aroma cartridge that attaches proximate to the outlet 1104 and which is configured to release an aroma into the purified air being discharged through the outlet 1104. The aroma cartridges are replaceable, and can include any of a variety of scents, such as pine, gardenia, menthol, vanilla, etc. Each aroma cartridge will preferably have a finite life, after which it will need to be replaced.

As shown in FIG. 6, some implementations of the HAIP 1000 can include one or more sensors 1180 that are located in a variety of locations about the HAIP 1000. The sensors 1180 can sense a variety of characteristics (e.g., air quality, temperature, carbon monoxide levels, etc.) associated with either the HAIP 1000 or the air that passes through the HAIP 1000. In addition, the sensors 1180 can send sensed data to a processor 1181 associated with the HAIP 1000. The processor 1181 can process and analyze the sensed data and, in turn, modify one or more parameters of the HAIP 1000 (e.g., fan speed, direction of air flow, etc.) in order to achieve a desired result. Additionally, the HAIP 1000 can include a warning system 1190 that can deliver a warning to a user based on the sensed data processed by the processor. The sensors 1180 can communicate either wirelessly or directly with the processor, and the processor can communicate either wirelessly or directly with the warning system 1190. The warning system 1190 can communicate in a variety of ways to the user, including directly from the air purification system (e.g., an audible alarm) or remotely (e.g., mobile alerts, etc.), as will be discussed in greater detail below. The user can also monitor the collected sensed data, as well as monitor and adjust one or more setting of the HAIP 1000 either directly or remotely (e.g., via an app downloaded onto the user's mobile device). The HAIP 1000 can include a wireless communication feature 1193 that can assist with providing wireless communication between the HAIP 1000 and remote devices.

As with other implementations of an air purifier or air purification and sensing system, such as that shown in FIG. 1, for example, the HAIP 1000 is more effective at sensing pollutants, pathogens, or noxious substances in the air because the systems actually cause the air to flow to or over/around the one or more sensors. This drastically reduces the time to sense, as compared to sensors that are statically-positioned in a room or other space. In other words, air with the substance or characteristic to be sensed is directed to the one or more sensors. Accordingly, any lag time to sense a part or characteristic of air is reduced.

Super Oxidation Purifier (SOP)

The SOP combines a number of technologies to most effectively destroy various contaminants in various gases and liquids, such as air and water, as described further below.

Reaction Chamber (RC)

The RC 1112 houses an ultraviolet (UV) light source, which can also produce ozone, as well as contains a coating that keeps maximum UV light within the UV-C range and to minimize loss of UV light to non-reflective surfaces. The RC 1112 also prevents UV light from escaping from the HAIP 1000, and is constructed to make impossible human exposure to the UV light. The RC 1112 is also designed to allow maximum airflow with minimal friction loss. In a preferred exemplary implementation, the air is pushed by the UV light source in a spiral fashion, which will allow the most even and consistent exposure of all air particles to the UV light. This spiral airflow can be achieved by cooperation between the axial fan 1108 and first radial louver 1110 at the inlet to the RC 1112. The axial fan 1108 moves the air in a spiral fashion with the rotation of fan's impeller, and the first radial louver 1110 deflects the air as it passes the axial fan 1108.

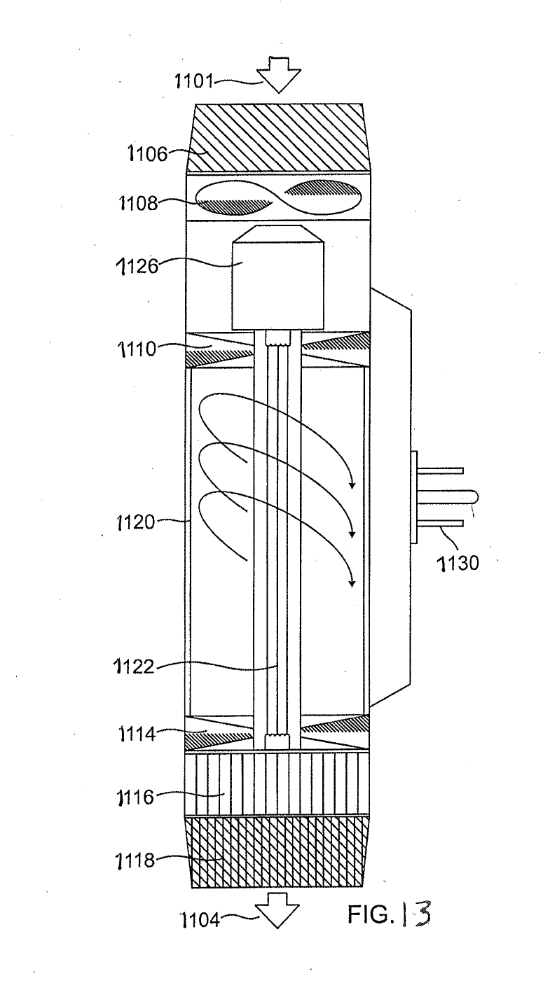

In some implementations, as shown in FIG. 13, the UV lamp ballast 1126 can be arranged after the axial fan 1108 and before the inlet radial louver 1110, for shielding of UV light from the UV light source 1122, and so as to not create a spiral forward air flow until just at the UV light source 1112. Also, this arrangement allows air to cross over and cool the UV lamp ballast in a laminar flow, rather than a spiral flow.

The RC 1112 is formed by at least part of the purifier housing 1102, which at least part is lined with a reflective material 1120 that is highly reflective to UV light, particularly in the UV-C range, and in some preferred implementations specifically in the 185 and 254 nanometer ranges. In one preferred implementation, the reflective material 1120 is a “lambertian” reflector, also known as a diffused reflector that reflects light at all angles to expose all air and contaminant molecules from all sides. Because of this high efficiency reflector, the HAIP 1000 can achieve high UV intensities in a smaller chamber than would otherwise be required in a conventional chamber.

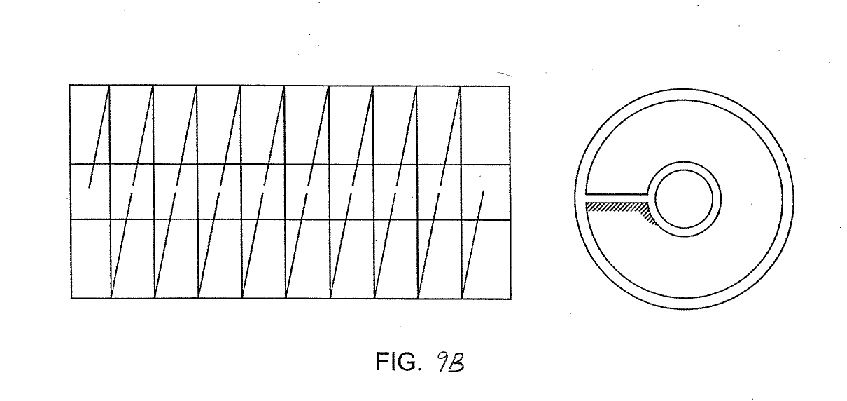

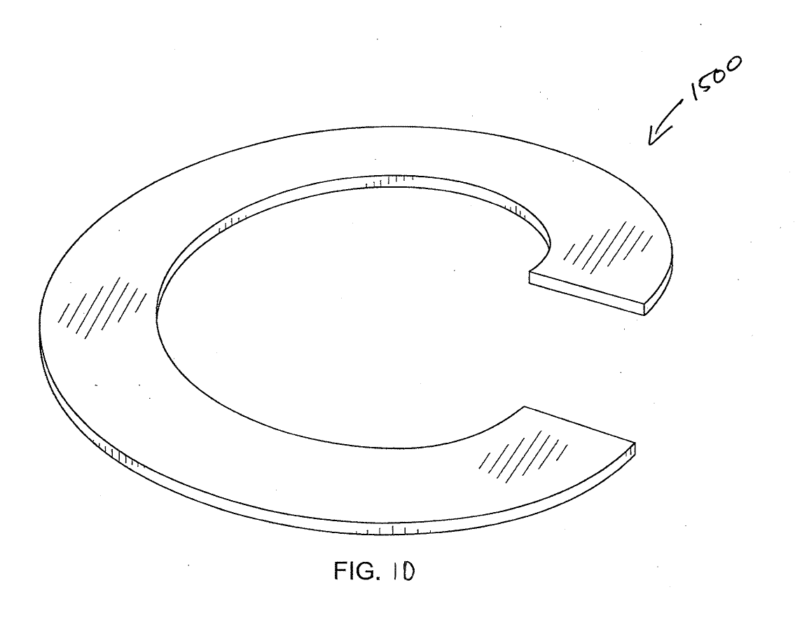

The RC 1112 housing can be constructed of metal, glass, ceramic, plastic, or the like, and coated with TiO₂ on the inside surface. The RC 1112 is formed to a shape or pattern maximize a surface area. FIG. 8 shows a star pattern chamber 1300, which has a number of angled peaks and valleys formed linearly along the length of the chamber and RC 1112 housing. FIGS. 9A and 9B show a continuous helical ramp chamber 1400. FIG. 10 shows a modular ramp chamber 1500.

The RC 1112 includes a UV light source 1122, which can either be ozone producing or non-ozone producing. The UV light source 1122 is preferably a low pressure mercury vapor lamp. In the ozone producing implementation, the light source 1122 produces light in the 254 nm (germicidal) range and in the 185 nm (ozone producing) range. The interaction between the two different wavelength ranges generates hydroxyl radicals, which are very powerful oxidizers that destroy many microbiological and chemical compounds. In the non-ozone producing implementation, the light source 1122 produces light primarily in the 254 nm (germicidal) range, which can destroy microorganisms such as viruses, bacteria, mold spores, parasites, etc. The UV light source 1122 is mounted in the RC 1112 such that it will not function should any attempt be made to remove it from the chamber.

The optional catalyst cartridge 1116 attaches to the outlet of the RC 1112, and is configured to convert ozone to oxygen. The capacity of the catalyst is preferably matched to the ozone production of the UV lamp to reduce ozone emissions from the HAIP 1000 to desirable levels. The catalyst cartridge 1116 can be constructed of an aluminum or ceramic substrate that is coated with a catalytic material, such as manganese dioxide, for instance.

Photo-Catalysis is defined as “acceleration by the presence of a catalyst”. A catalyst does not change in itself or being consumed in the chemical reaction. This definition includes photosensitization, a process by which a photochemical alteration occurs in one molecular entity as a result of initial absorption of radiation by another molecular entity called the photosensitized. Chlorophyll of plants is a type of photo catalyst. Photo catalysis compared to photosynthesis, in which chlorophyll captures sunlight to turn water and carbon dioxide into oxygen and glucose, photo catalysis creates strong oxidation agent to breakdown any organic matter to carbon dioxide and water in the presence of photo catalyst, light and water.

Mechanism of Photo-Catalysis

When photo catalyst titanium dioxide (T₁O₂) absorbs Ultraviolet (UV) radiation from sunlight or illuminated light source (fluorescent lamps), it will produce pairs of electrons and holes. The electron of the valence band of titanium dioxide becomes excited when illuminated by light. The excess energy of this excited electron promoted the electron to the conduction band of titanium dioxide therefore creating the negative-electron (e−) and positive-hole (h+) pair. This stage is referred as the semiconductor's ‘photo-excitation’ state. The energy difference between the valence band and conduction band is known as the ‘Band Gap’. Wavelength of the light necessary for photo-excitation is: 1240 (Planck's constant, h)/3.2 ev (band gap energy)=388 nm.

Sterilizing Effect

Photo catalyst does not only kill bacteria cells, but also decompose the cell itself. The titanium dioxide photo catalyst has been found to be more effective than any other antibacterial agent, because the photo catalytic reaction works even when there are cells covering the surface and while the bacteria are actively propagating. The end toxin produced at the death of cell is also expected to be decomposed by the photo catalytic action. Titanium dioxide does not deteriorate and it shows a long-term anti-bacterial effect. Generally speaking, disinfections by titanium oxide are three times stronger than chlorine, and 1.5 times stronger than ozone.

Deodorizing Effect

On the deodorizing application, the hydroxyl radicals accelerate the breakdown of any Volatile Organic Compounds or VOCs by destroying the molecular bonds. This will help combine the organic gases to form a single molecule that is no harmful to humans thus enhance the air cleaning efficiency. Some of the examples of odor molecules are: Tobacco odor, formaldehyde, nitrogen dioxide, urine and fecal odor, gasoline, and many other hydrocarbon molecules in the atmosphere. Air purifier with T102 can prevent smoke and soil, pollen, bacteria, virus and harmful gas as well as seize the free bacteria in the air by filtering percentage of 99.9% with the help of the highly oxidizing effect of photo catalyst (T102).

Air Purifying Effect

The photo catalytic reactivity of titanium oxides can be applied for the reduction or elimination of polluted compounds in air such as NOx, cigarette smoke, as well as volatile compounds arising from various construction materials. Also, high photo catalytic reactivity can be applied to protect lamp-houses and walls in tunneling, as well as to prevent white tents from becoming sooty and dark. Atmospheric constituents such as chlorofluorocarbons (CFCs) and CFC substitutes, greenhouse gases, and nitrogenous and sulfurous compounds undergo photochemical reactions either directly or indirectly in the presence of sunlight. In a polluted area, these pollutants can eventually be removed.

Water Purification

Photo catalyst coupled with UV lights can oxidize organic pollutants into nontoxic materials, such as CO2 and water and can disinfect certain bacteria. This technology is very effective at removing further hazardous organic compounds (TOCs) and at killing a variety of bacteria and some viruses in the secondary wastewater treatment. Pilot projects demonstrated that photo catalytic detoxification systems could effectively kill fecal coli form bacteria in secondary wastewater treatment.

Controller

An electronic housing 1124 houses an electronic control module and controller circuit. The electronic control module includes a lamp ballast 1126. The lamp ballast 1126 can be an alternating current (AC) ballast that plugs directly into a household electrical outlet for typical 100-240 VAC. Alternatively, the lamp ballast 1126 can be a direct current (DC) ballast that will typically work on 12 VDC. The DC ballast version of the HAIP 1000 is designed for desktop units, portable units, automotive, recreational vehicle, and boat use, as just some examples. The DC ballast is described further below. In yet another alternative, the lamp ballast 126 can be a universal serial bus (USB) powered ballast, which can be connected to a USB port of a laptop or desktop computer to provide a user with clean air.

The electronic control module incorporates the axial fan 1108 that moves air through the various air purification components within the unit, as described above. In some implementations, the axial fan 1108 is variable speed. On a high-speed setting, the axial fan 1108 moves more air through the air purifier for greater efficiency, but will also generate more noise. A low-speed setting may be preferred for a quiet room such as a bedroom or for night use. In some implementations, the HAIP 1000 can include a manual controller for controlling the fan speed. In other implementations, the HAIP 1000 can include an automatic mode, by which fan speed can be controlled by a light sensor. For example, the HAIP 1000 can be run on a “nighttime/quiet mode” that will run the axial fan 1108 at low speed during the night, or the HAIP 1000 can be run on a “daytime/quiet mode” that will run the axial fan 1108 at low speed during the day.

The HAIP 1000 can also include a light or series of lights incorporated into the housing 1102 that indicate operation of the device. The lights can be programmed to gently pulsate or wave during normal operation. Optionally, a light or lights can be set to operate as a nightlight. The light sensor can be used to activate the nightlight light or lights during darkness.

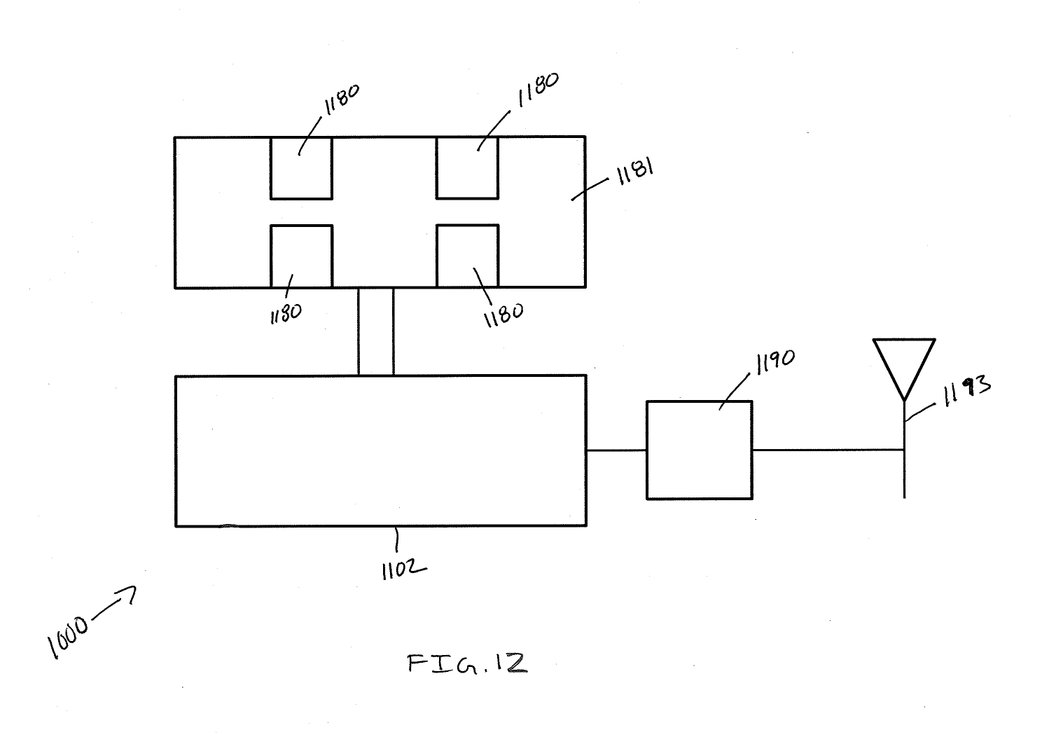

FIG. 12 illustrates the HAIP 1000 including a processor 1181 and sensors 1180 that are in communication with the processor 1180. This can allow the processor to process and analyze the sensed data collected from the sensors 1180. In addition, the HAIP 1000 can include a wireless communication feature 1192, such as an antenna 1182. The wireless communication feature can be in communication with the warning system 1190 for allowing alerts to be sent remotely, such as to a mobile device associated with a user. In addition, the wireless communication feature can be in communication with the processor, such as for allowing the wireless transmission of data and instructions between a remote device, such as the user's mobile device, and the HAIP 1000. This can allow the user to monitor the sensed data collected by the HAIP 1000, as well as monitor and adjust settings associated with the HAIP 1000.

In some implementations, the HAIP 1000 can wireless transmit signals representing sensed data or information to a mobile device. The mobile device can include a local application for receiving, interpreting, and displaying information related to the sensed data, as well as controls for receiving user input commands that can be wirelessly communicated back to the HAIP 1000 via a communication network, to control an operation of the HAIP 1000. For instance, if a sensor senses smoke from an outside air source, the mobile device application can generate a rendering of an alarm, and allow the user to remotely and wirelessly reduce the airflow from an environment outside a building to the inside environment, effectively shutting off a pathway for the smoke. Other signals and warnings are possible, and other control signals may be employed, such as, without limitation, air flow rate, temperature, UV light intensity, and/or other electro-mechanical operations such as louvers, fans, light ballasts, etc.

Although a few embodiments have been described in detail above, other modifications are possible. For instance, the inlet 1101 and/or outlet 1104 of the HAIP 1000 can include a directionally changeable nozzle or some other dynamically adjustable device for providing a wider range of inlet and outlet directionality. Other embodiments may be within the scope of the following claims.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An air purification system comprising:
a housing including an air inlet and an air outlet;
a fan actuated by a control circuit that controls a rate of airflow through the air purification system;
a filter for filtering out particulates from the air passing through the housing;
an ultraviolet light source providing ultraviolet light to the air passing through the housing;
at least one photo-catalytic element positioned adjacent the ultraviolet light source;
a chemical catalyst element that is exposed to the air passing through the housing; and
a sensor for collecting sensed data defining one or more characteristic associated with the air passing through the housing.

2. The air purification system of claim 1, further comprising a processor configured to compare the sensed data with an acceptable range.

3. The air purification system of claim 2, further comprising a warning system that is configured to provide an alarm to a user when the processor determines that the sensed data is not within the acceptable range.

4. The air purification system of claim 3, further comprising a wireless communication feature that is in communication with at least one of the processor and the warning system.

5. The air purification system of claim 4, wherein the wireless communication feature is configured to send at least one of the alarm, the sensed data, and a setting of the air purification system to a remote device.

6. The air purification system of claim 5, wherein the remote device includes at least one of a mobile device and a computer.

7. The air purification system of claim 5, wherein the wireless communication feature is configured to receive an instruction from the remote location, the instruction comprising a change to the setting of the air purification system.

8. The air purification system of claim 2, wherein the processor is further configured to change a setting of the air purification system based on the comparison of the sensed data.

9. The air purification system of claim 1, wherein the sensor includes a temperature gauge configured to collect sensed data defining a temperature of the air passing through the air purification system.

10. The air purification system of claim 1, wherein the sensor includes a smoke detector configured to collect sensed data defining an amount of smoke in the air passing through the air purification system.

11. The air purification system of claim 1, wherein the sensor includes a carbon monoxide detector configured to collect sensed data defining an amount of carbon monoxide in the air passing through air purification system.

12. A method, comprising:
sensing, with a first sensor, a first characteristic of air adjacent a first side of a housing of an air purification system, the air purification system being configured to purify air passing through the housing;
determining, by a processor of the air purification system, whether the first characteristic is within an accepted first range; and
changing, when the first characteristic is determined to not be within the accepted first range, a setting associated with the air purification system to assist the first characteristic with falling within the accepted first range.

13. The method of claim 12, further comprising:
sensing, with a second sensor, a second characteristic of air adjacent a second side of the housing of the air purification system;
calculating, by the processor, a difference between the first characteristic and the second characteristic;
determining, by the processor, if the calculated difference is within an accepted second range; and
changing, when the calculated difference is determined to not be within the accepted second range, the setting associated with the air purification system to assist the calculated difference with falling within the accepted first range.

14. The method of claim 12, wherein the setting includes a fan speed of a fan configured to control a speed at which the air passes through the housing.

15. The method of claim 12, wherein the first characteristic includes a temperature, a pressure, an amount of smoke in the air, and an amount of carbon monoxide in the air.

16. The method of claim 12, further comprising:
activating, based on the determining, a warning system of the air purification system.

17. The method of claim 16, wherein the activating the warning system includes at least one of activating an audible alarm and sending an alert to a remote device.

18. The method of claim 13, further comprising:
sending, from a wireless communication feature of the air purification system in wireless communication with a remote device, information related to at least one of the first characteristic and the second characteristic to the remote device.

19. The method of claim 18, further comprising:
receiving, at the wireless communication feature, a setting instruction from the remote device; and
changing, based on the setting instruction, the setting of the air purification system.

20. The method of claim 13, wherein the second side of the housing is located outside of a structure to which the air purification system is coupled to and the first side of the housing is located at least one of inside the housing and inside the structure to which the air purification system is coupled to.
